(19) United States
(12) Patent Application Publication
(10) Pub. No.: US 2004/0109886 A1
(43) Pub. Date: Jun. 10, 2004

(54) Methods and apparatus for transdermal delivery of abusable drugs with a deterrent agent

(76) Inventor: Larry Rigby, Salt Lake City, UT (US)

Correspondence Address: KIRTON AND MCCONKIE, 1800 EAGLE GATE TOWER, 60 EAST SOUTH TEMPLE, P O BOX 45120, SALT LAKE CITY, UT, 84145-0120, US

(21) Appl. No.: 10/649,322

(22) Filed: Aug. 27, 2003

Related U.S. Application Data
(60) Provisional application No. 60/406,288, filed on Aug. 27, 2002.

(51) Int. Cl. A61K 31/445; A61K 9/70

(52) U.S. Cl. 424/449; 514/317

(57) ABSTRACT

The present invention describes a system and method for reducing the abuse potential of drugs, particularly or especially narcotic agents, in transdermal drug delivery systems. This is achieved by designing the transdermal drug delivery system such that when the active drug is extracted out of the transdermal delivery system, a deterrent agent is also timely co-extracted upon introduction of the system to an extraction solution where abuse may normally be allowed take place. Preferably, the deterrent agent of the present invention is capable of inducing or causing one or more intensely repugnant effects within the abusing person, thus reducing the potential for abuse of the active drug formulation.

### PRIORITY DOCUMENTS

This application claims priority to United States provisional application serial number 06406,288, filed Aug. 27, 2002, entitled METHODS AND APPARATUS FOR TRANSDERMAL DELIVERY OF ABUSABLE DRUGS WITH A DETERRENT AGENT.

### BACKGROUND

1. Field of the Invention

The present invention relates to the systemic delivery of active drug agents or compounds via transdermal drug delivery methods. Particularly, the present invention relates to a transdermal drug delivery system, wherein abusable drugs serve as the active drug agent, and wherein a deterrent agent is utilized to reduce the potential for abuse of the abusable drug.

2. Background of the Invention and Related Art

Many drugs, including those that have abuse potentials, such as narcotic agents, may be delivered through transdermal absorption methods to provide unique therapeutic effects. This type of drug delivery, in order to maintain a sufficient transdermal permeation driving force throughout the application, usually requires a large amount of active drug to be present in the formulation. As a result, there is often a large amount of active drug still present in the formulation upon completion of the application. The presence of this residual active drug source provides a potential for abuse. Indeed, abuse of this drug may be made either by extracting it out of the used or new formulation or by other methods, such as snorting or oral transmucosal absorption (much faster than transdermal). Many substances may be abused this way, including but not limited to fentanyl, sufentanil, and other mu agonists.

In attempting to address this issue, U.S. Pat. No. 5,236,714 to Lee et al. discloses the use of antagonist agents in the formulation or in the delivery device to reduce the abuse potential of the drug source. However, antagonists are usually quite expensive and can interfere with the intended drug delivery process. For instance, if the antagonist and the active drug are in the same formulation, some quantities of the antagonist may be delivered into the circulation systemically, along with the active drug, thus potentially compromising the desired pain control effected by the active drug. More importantly, the antagonist only negates the effect of the abusable drug. It does not give the abuser a painful or unpleasant experience which would give the abuser stronger reason not to abuse the product again.

Accordingly, what is needed is an improved way to reduce the abuse potential of transdermal drug delivery systems, and particularly transdermal narcotic delivery systems.

### SUMMARY AND OBJECTS OF THE INVENTION

Transdermal drug delivery systems provide an effective way to administer an active drug to a patient under a controlled environment. The present invention contemplates providing controlled active drug delivery of narcotic agents through a transdermal delivery apparatus and method, while also providing for a reduced potential for abuse of the active drug.

In accordance with the invention as embodied and broadly described herein, the present invention features a transdermal drug delivery system comprising (a) an active drug formulation contained within a carrier medium, wherein the active drug formulation comprises an abusable drug that may be systemically circulated within a user; (b) means for preventing the delivery of said deterrent agent into the user when the active drug formulation is systemically applied; and (c) a deterrent agent also contained within the carrier medium, and said deterrent is co-extracted with the active drug when the system is subject to an extraction solution or is co-administered with the active drug when the system is used on mucosal surfaces (i.e. oral transmucosal absorption or chewing in the mouth). In essence, the present invention describes a system and method for reducing the abuse potential of drugs, particularly or especially narcotic agents, in transdermal drug delivery systems. This is achieved by designing the transdermal drug delivery system such that when the active drug is extracted out of the transdermal delivery system, a deterrent agent is also timely co-extracted upon introduction of the system to an extraction solution where abuse may normally be allowed to take place. Preferably, the deterrent agent of the present invention is capable of inducing or causing one or more intensely unpleasant effects within the abusing person, thus minimizing the potential for abuse of the active drug formulation.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates a transdermal drug delivery system comprising a backing film separating the active drug formulation from the deterrent agent according to a preferred embodiment of the present invention;

FIG. 2 illustrates a transdermal drug delivery system comprising a backing film having a deterrent agent applied thereon and an active drug formulation applied on top of the deterrent agent with an optional film separating the two according to an alternative embodiment of the present invention; and

FIG. 3 illustrates a transdermal drug delivery system comprising a backing film having an active drug formulation applied thereon and a plurality of microencapsulated deterrent agents supplied within the active drug formulation.

### DETAILED DESCRIPTION OF THE PREFERRED EMBODIMENTS

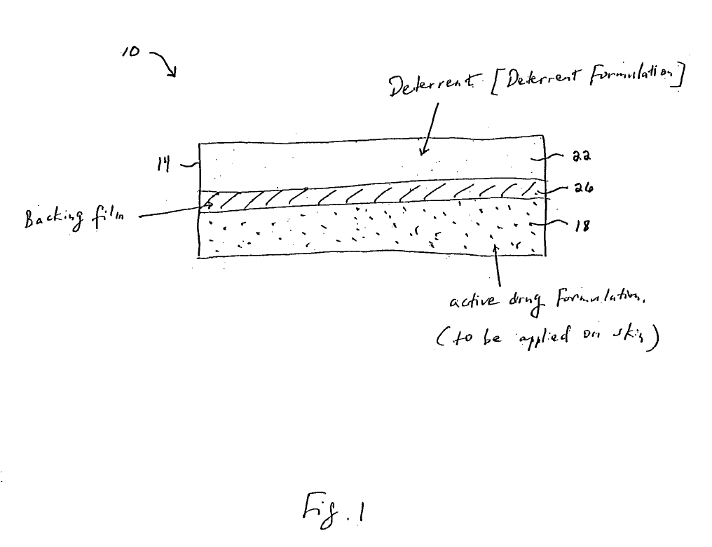

It will be readily understood that the components of the present invention, as generally described and illustrated in the figures herein, could be arranged and designed in a wide variety of different configurations. Thus, the following more detailed description of the embodiments of the system and method of the present invention, and represented in FIGS. 1 through 3, is not intended to limit the scope of the invention, as claimed, but is merely representative of the presently preferred embodiments of the invention.

The presently preferred embodiments of the invention will be best understood by reference to the drawings wherein like parts are designated by like numerals throughout.

The present invention describes a method and system for reducing the abuse potential of drugs, and particularly or especially narcotic agents, in transdermal drug delivery systems. This is achieved by designing the transdermal delivery system such that when the active drug is extracted out of the transdermal delivery system, a deterrent agent is simultaneously co-extracted. Another design is that when the transdermal delivery system is used by the abuser in an unintended way to obtain a quicker absorption of the abusable drug, such as snorting or oral transmucosal delivery, the deterrent is co-administered with the abusable drug. Preferably, the deterrent agent of the present invention is capable of inducing or causing one or more intensely unpleasant effects, unrelated to the effects of the active drug itself, within the abusing person, thus reducing the potential for abuse of the active drug in the system. Stated differently, the deterrent agent existing within the transdermal drug delivery system functions to repulse the abuser by inducing violent and/or offensive bodily reactions, unrelated to the effects of the active drug itself, upon improper use of the transdermal drug delivery system. So severe are these reactions that the abuser is highly motivated thereafter to deliberately avoid such abuse in the future. Indeed, the presence of such a deterrent and associated repulsive effects serves to curtail any use of the drug beyond that which is appropriately prescribed, thus reducing the potential for abuse of the active drug. The severely unpleasant experience with the deterrent agent is worse than a mere minimized euphoria or at best a dissapointment of no euphoria that an antagonist can cause. Therefore, the inventor believes the abuse potential in the transdermal abusable drug delivery systems can be reduced more with the use of the deterrent, as provided in this invention, than systems that involves antagonist(s).

The present invention contemplates the use of several possible deterrent agents, all of which are not and cannot be recited herein. One of ordinary skill in the art will recognize the many possible agents that may be used as deterrent agents in the formulation of the present invention transdermal drug delivery system. While all the possible deterrent agents are impossible to recite herein, it should be noted that the deterrent agents may be compounds, chemicals, etc. that have one or more of the following properties: (1) can cause severe irritation when injected (i.e. Capsaicin); (2) can cause mood depression (i.e. droperidol) or other pronounced central nervous system (CNS) effects; (3) can cause acute gastrointestinal, cardiac or respiratory effects; (4) can cause violent nausea or vomiting; (5) can elicit unpalatable bitterness or other repulsive tastes in the mouth; (6) can produce repugnant smells if not used as instructed; and/or (7) can cause the abuser to fall asleep rapidly, thus causing him to miss or be made unaware of the euphoria.

As the deterrent agent is capable of causing or inducing one or more intensely unpleasant effects if delivered to the body or placed in the mouth, the structure of the transdermal drug delivery system is designed so that the deterrent agent is not delivered into the body when the transdermal delivery system is used by the patient as intended, but only when used in unprescribed ways and the potential for abuse exists. The present invention contemplates several designs or embodiments for controlling the delivery and extraction of both the active drug and the deterrent agent as appropriate.

With reference to FIG. 1, the preferred embodiment, the present invention transdermal drug delivery system 10 comprises a carrier medium 14, such as a transdermal patch, an active drug formulation 18, a deterrent agent 22, and a backing film 26 separating active drug formulation 18 from deterrent agent 22. Active drug formulation 18 is placed on one side of backing film 26, with deterrent agent (or deterrent formulation) 22 coated on the other side of backing film 26, opposite active drug formulation 18, so that deterrent agent 22 and active drug formulation 18 are completely separated by backing film 26. Carrier medium 14 is designed to comprise an adhesive on the side containing active drug formulation 18 so that a proper amount of the drug can be delivered through transdermal permeation if the formulation is applied to human skin. In contradistinction, carrier medium 14 is designed to comprise a non-adhesive. Therefore, if by chance the transdermal drug delivery system 10 placed in an extraction solution for the purpose of extracting out more of the active drug formulation 18 (i.e. for the purpose of abusing the system), deterrent agent 22 will also be extracted into the solution where it may become active in inducing the extremely negative and unpleasant effects if introduced into the body. If this system is placed in the mouth for obtaining fast absorption of the abusable drug, the deterrent will be in contact with and enter the saliva to cause the unpleasant effect.

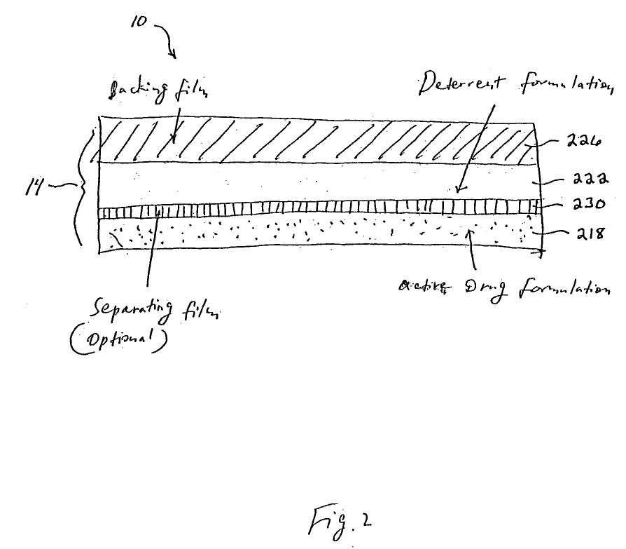

FIG. 2 represents an alternative embodiment for appropriately controlling the delivery and extraction of both the active drug and the deterrent agent. Specifically, FIG. 2 illustrates transdermal drug delivery system 10 comprising a carrier medium 14 containing an active drug formulation 218, a deterrent agent or formulation 222, a backing film 226, and a separating film 230. In this design, a layer of deterrent formulation 222 is placed between active drug formulation 218 backing film 226, with deterrent formulation 222 and active drug formulation 218 being separated by a separating film 230. Separating film 230 is a good barrier to deterrent agent 222, but dissolves in the common extraction solutions used to extract active drug formulation 218. When transdermal drug delivery system 10 is placed in an extraction solution (or in contact with salive when placed in the mouth), active drug formulation 218 and deterrent formulation 222 will both dissolve into the solution or saliva once separating film 230 dissolves in the solution. It should be noted that in this design the use of separating film 230 is optional. It is possible to use a deterrent formulation that prohibits the movement of the deterrent agent into the active drug formulation, thus avoiding the use of the separating film. For example, if the deterrent agent is fixed in a layer of dried soluble starch polymer, most of the deterrent will be unable to move into the formulation layer and subsequently the skin.

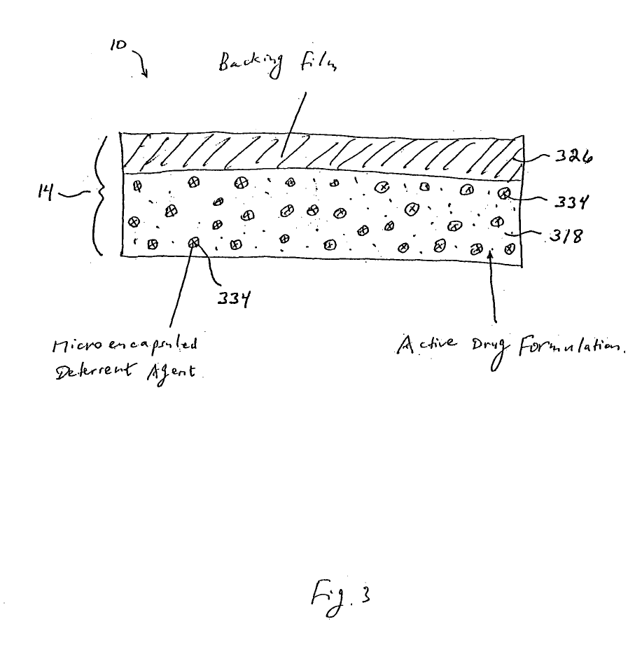

With reference to FIG. 3, shown is a third alternative embodiment of a transdermal drug delivery system 10 comprising an active drug formulation 318, a backing film 326, and a plurality of microencapsulated deterrent agents 334. In this design, deterrent agent 322 is microencapsulated with a material that is not soluble in the solvent used in manufacturing the drug formulation, but is however, soluble in common solutions that may be used for extraction of active drug formulation 318. For example, acidic water or alcohol may be used for extracting fentanyl from a silicone glue matrix formulation, and n-heptane may be used as solvent in manufacturing the silicone formulation. As such, the microencapsulation material must be insoluble in n-heptane and soluble in acidic water or alcohol. When the transdermal drug delivery system 10 is placed into the extraction solution, the microcapsules dissolve and release the deterrent agent into the extraction solution, thus reducing the potential for abuse of the active drug formulation 218 existing within transdermal drug delivery system 10.

In a fourth embodiment, transdermal drug delivery system 10 is equipped with a deterrent agent that is extremely bitter to the taste, or that has another associated unpalatable taste, either in the formulation or coated in the back of the backing film. Many compounds can cause bitterness, including many alkaloids, such as berberine and its derivatives (i.e. berberine sulfate). The bitterness or the bad taste cannot be sensed by the skin when the patch is used as intended. However, if the patch is placed in the mouth to obtain a quick delivery of the active drug through the oral mucosal absorption, the agent is released into the mouth via saliva and causes an unpleasant bitterness or bad taste in the mouth, thus likewise reducing the potential for abuse.

In yet another alternative embodiment, the present invention features an oral transdermal drug delivery system, wherein if the transdermal delivery system is placed in the mouth to obtain a more rapid release and pain relieving effect, a similar deterrent agent is also released into the mouth.

While the above-described embodiments represent several different designs, they are not meant to be limiting in any way. Indeed, other forms, designs, structures, compounds, delivery mechanisms, etc. are intended and are contemplated by the present invention as will be recognized by and apparent to one of ordinary skill in the art. In addition, while the above embodiments describe several designs, the present invention contemplates the utilization or incorporation of one or several of the above described designs into a single transdermal drug delivery system. As such, one of ordinary skill in the art will recognize the many and several design configurations that may be employed into a transdermal drug delivery system according to the spirit of the invention as described, shown, and claimed herein.

The following Examples are provided to set forth and illustrate actual designs of several transdermal drug delivery systems comprising the active drug formulation, the deterrent agent and formulations, and the delivery mechanisms used to deliver and release such. These examples are merely illustrative and are not to be construed as limiting in any way. Indeed, one ordinarily skilled in the art will recognize other ways to practice the present invention as intended herein.

### EXAMPLE ONE

A transdermal fentanyl drug delivery system (fentanyl patch) is back coated with a droperidol deterrent agent. A silicone glue containing a fentanyl base formulation (drug formulation) is coated to one side (drug side) of a plastic film. The finished drug formulation has 0.3 mg of fentanyl per cm2. And the dried fentanyl-in-silicone glue is capable of delivering fentanyl into a human body's systemic circulation when applied to the skin, with a flux of approximately 2.5 mcg/hr/cm2. The other side of the film is coated with 5 mg/cm2 of a droperidol deterrent agent in a gelatin formulation (deterrent formulation) that is soluble in water. There are two easily obtainable solvents for extracting fentanyl out of the drug formulation: acidic water and alcohol. If this patch is placed in acidic water, droperidol being a base with pKa of 7.64 and is soluble in acidic water, will be co-extracted into the acidic water because gelatin dissolves in water. If this is placed in alcohol or an alcohol-water mixture, droperidol will also be extracted out because droperidol exhibits high solubility in alcohol. The administration of droperidol with fentanyl is likely to elicit one or more of the following known side effects of droperidol, each of which will undoubtedly cause the potential abuser to feel miserable: intense nausea or loss of appetite; constipation; drowsiness; dizziness; acute headaches; loss of libido; and anxiety. Such effects will greatly reduce the abuse potential of the fentanyl patch.

### EXAMPLE 2

A fentanyl patch similar to that in Example 1, except that the deterrent formulation is sandwiched between the backing film and the drug formulation.

### EXAMPLE 3

A fentanyl patch with a microencapsulated droperidol deterrent agent. In this system, the fentanyl is contained in a silicone glue formulation coated on a backing film. Microcapsules of droperidol are also mixed in the formulation. The material used to form the envelope of the microcapsules is soluble in acidic water and alcohol, but not appreciably so in the silicone glue formulation and not so acidic water. When this patch is placed into an extraction solvent such as acidic water or alcohol, the microcapsules will open and release the droperidol into the extraction solution. However, if this patch is placed on skin as intended, the microcapsules do not break and thus no droperidol is released into the skin. Because the encapsulating material is not soluble in acidic or semi-acidic water, even a small amount of sweat into the formulation will not open the microcapsules.

### EXAMPLE 4

Similar to Examples 2 and 3, except that the deterrent agent is capsaicin. The fentanyl solution containing capsaicin will cause a torturous burning sensation if snorted or inhaled, thus significantly reducing the abuse potential of the transdermal drug delivery system.

### EXAMPLE 5

Similar to Examples 2 and 3, except that the deterrent is an agent that can cause severe nausea if injected, snorted, or inhaled, such as an emetic. The following emetics can be used as well as others not listed: the syrup of ipecac (ipecacuanha), sulfate of zinc or copper, alum, ammonium carbonate, mustard in water, copious quantities of warm saltwater, cardiac glycosides, dihydroxyphenylalanine (L-DOPA) and apomorphine.

### EXAMPLE 6

Similar to Examples 2 and 3, except that the deterrent is an agent that can cause the abuser to fall asleep rapidly if injected, snorted, or inhaled. The resultant effect of this particular deterrent will be that the potential abuser will either fall asleep and/or be unaware of the euphoric effects experienced from the active fentanyl drug.

### EXAMPLE 7

A fentanyl transdermal drug delivery patch is provided with reduced mucosal absorption abuse potential. A fentanyl formulation similar to that in Example 1 is coated on one side of a backing film. A deterrent agent or formulation containing berberine sulfate is coated on the other side of the backing film. If the abuser places a piece of this patch into his/her mouth in an attempt to obtain a rapid absorption of fentanyl, the berberine sulfate is released into the saliva and the mouth, which causes a very bitter and highly pronounced unpalatable taste in the potential abusers mouth. This will most likely cause the potential abuser to spit out the patch, thus reducing the potential for abuse.

### EXAMPLE 8

A deterrent agent berberine sulfate is added into a silicone glue based fentanyl formulation containing similar amount of fentanyl as that in Example 1. The formulation is coated onto one side of the backing film. When this patch is used as intended, only trace amounts of berberine, if any, is absorbed through skin and causes no adverse side effect. But when a piece of this patch is placed in the mouth, berberine is subsequently released from the formulation into the mouth and causes an intensely bitter and highly repugnant taste, thus reducing the potential for abuse.

The present invention may be embodied in other specific forms without departing from its spirit of essential characteristics. The described embodiments are to be considered in all respects only al illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A transdermal drug delivery system comprising:
an active drug formulation contained within a transdermal patch, said active drug formulation comprising an abusable drug that is systemically administered to a user;
a deterrent agent also contained within said transdermal patch, said deterrent agent being co-extracted with said active drug formulation upon introduction of said system to an extraction solution; and
means for preventing the delivery of said deterrent agent into human skin when said active drug formulation is applied on skin.

2. The transdermal drug delivery system of claim 1, wherein said abusable drug is a narcotic agent.

3. The transdermal drug delivery system of claim 1, wherein said abusable drug is fentanyl.

4. The transdermal drug delivery system of claim 1, wherein said deterrent agent is selected from the group consisting of irritating agents, central nervous system effecting agents, gastrointestinal system effecting agents, cardiac effecting agents, respiratory system effecting agents, bowel effecting agents, palate effecting agents, sleep inducing agents, and smell effecting agents.

5. The transdermal drug delivery system of claim 1, wherein said abusable drug has the potential to be abused by inhalation.

6. The transdermal drug delivery system of claim 1, wherein said abusable drug has the potential to be abused by injection.e transdermal drug delivery system of claim 1, wherein said abusable drug has the potential to be abused by snortingl drug delivery system of claim 1, wherein said abusable drug has the potential to be abused by ingestion.

7. A transdermal drug delivery system comprising:
an active drug formulation comprising an abusable drug, said active drug formulation being coated on one side of a backing film; and
a deterrent formulation, said deterrent formulation being coated on the other side of said backing film.

8. A transdermal drug delivery system comprising:
a deterrent formulation coated on one side of a backing film; and
an active drug formulation coated on top of said deterrent formulation, said active drug formulation comprising an abusable drug.

9. The transdermal drug delivery system of claim 7, wherein said abusable drug is a narcotic agent.

10. The transdermal drug delivery system of claim 7, wherein said abusable drug is fentanyl.

11. The transdermal drug delivery system of claim 7, wherein said deterrent agent is selected from the group consisting of irritating agents, central nervous system effecting agents, gastrointestinal system effecting agents, cardiac effecting agents, respiratory system effecting agents, bowel effecting agents, palate effecting agents, sleep inducing agents, and smell effecting agents.

12. The transdermal drug delivery system of claim 7, further comprising a separating film separating said active drug formulation from said deterrent agent.

13. A transdermal drug delivery system comprising:
a carrier medium;
a drug formulation contained within said carrier medium and comprising an abusable drug; and
a microencapsulated deterrent agent also contained within said carrier medium;

14. The transdermal drug delivery system of claim 13, wherein said abusable drug is a narcotic agent.

15. The transdermal drug delivery system of claim 13, wherein said abusable drug is fentanyl.

16. The transdermal drug delivery system of claim 15, wherein said deterrent agent is selected from the group consisting of irritating agents, central nervous system effecting agents, gastrointestinal system effecting agents, cardiac effecting agents, respiratory system effecting agents, bowel effecting agents, palate effecting agents, sleep inducing agents, and smell effecting agents

17. The transdermal drug delivery system of claim 13, wherein the deterrent is an emetic.
